(19) United States
(12) Patent Application Publication
(10) Pub. No.: US 2016/0074519 A1
(43) Pub. Date: Mar. 17, 2016

(54) CROSSLINKED HYALURONIC ACID COMPOSITIONS

(71) Applicant: ALURON BIOPHARMA INC., Montreal (CA)

(72) Inventors: Vladimir KHABAROV, Moscow (RU); Mikhail SELYANIN, Moscow (RU); Felix POLYAK, Montreal (CA)

(21) Appl. No.: 14/786,906

(22) PCT Filed: Apr. 25, 2014
(86) PCT No.: PCT/CA2014/000378

§ 371 Date: Oct. 23, 2015

Related U.S. Application Data
(60) Provisional application No. 61/815,983, filed on Apr. 25, 2013.
(60) Provisional application No. 61/826,890, filed on May 23, 2013.

(51) Int. Cl. A61K 47/36; A61K 31/198; A61K 31/194; A61Q 19/08; A61K 8/73; A61K 8/67; A61K 8/41; A61K 8/362; A61K 31/519; A61K 31/375

(52) U.S. Cl. CPC A61K 47/36; A61K 31/519; A61K 31/198; A61K 31/194; A61K 31/375; A61K 8/735; A61K 8/673; A61K 8/41; A61K 8/362; A61K 8/676; A61Q 19/08; A61K 2800/40

(57) ABSTRACT

Crosslinked hyaluronic acid compositions are disclosed herein. More specifically, the hyaluronic acid is crosslinked with a biologically active compound selected from amino acids, amino esters, hydroxy acids, hydroxy esters, vitamins, stabilizers and mixtures thereof. The crosslinking is conveniently achieved by means of an extrusion process.

### CROSS-REFERENCE TO RELATED APPLICATIONS

The present application claims the benefit of priority from U.S. Provisional Applications Nos. 61/815,983 and 61/826,890 filed on Apr. 25, 2013 and May 23, 2013 respectively. The entire text of the above-referenced disclosures is specifically incorporated herein by reference.

### FIELD

The present disclosure broadly relates to hyaluronic acid compositions. More specifically, but not exclusively, the present disclosure relates to crosslinked hyaluronic acid compositions. The present disclosure also relates to a process for the production of the crosslinked hyaluronic acid compositions.

### BACKGROUND

Hyaluronic acid (HA) is a naturally occurring polyanionic, non-sulfated glycosaminoglycan that consists of N-acetyl-D-glucosamine and β-glucoronic acid. It is present in the intercellular matrix of most vertebrate connective tissues especially skin where it has a protective, structure stabilizing and shock-absorbing role. As skin ages and is repeatedly exposed to the sun's ultra violet rays, dermal cells decrease their production of hyaluronic acid and increase the rate of its degradation. Likewise, aging skin loses collagen and elastin, other natural substances necessary to keep skin youthful and resilient. Over time, the loss of hyaluronic acid, collagen and elastin causes aging skin to develop lines, wrinkles, and folds.

The unique viscoelastic nature of HA along with its biocompatibility and non-immunogenicity has led to its use in a number of clinical applications, which include: the supplementation of joint fluid in arthritis; as a surgical aid in eye surgery; and to facilitate the healing and regeneration of surgical wounds. More recently, HA has been investigated as a drug delivery agent for various routes of administration, including ophthalmic, nasal, pulmonary, parenteral and topical.

In the past several years, compositions of hyaluronic acid have been used in cosmetic applications to fill wrinkles, lines, folds, scars, and to enhance dermal tissue. Because hyaluronic acid is natural to the human body, it is a generally well tolerated and fairly low risk skin augmentation product. Originally, hyaluronic acid compositions contained particles, or microspheres, of hyaluronic acid suspended in a gel. These compositions, which are still in commercial use, tend to degrade within a few months after injection and thus require fairly frequent reinjection to maintain their skin augmenting effect. Specifically, hyaluronic acid is highly soluble in its natural state and has a rapid turnover through enzymatic and free radical metabolization.

More recently, compositions of crosslinked hyaluronic acid have been used for dermal augmentation. These hyaluronic acid compositions are typically crosslinked with bifunctional crosslinking agents to form a less water soluble polymer hydrogel network that is more resistant to degradation, and thus requires less frequent reinjection, than the non-crosslinked hyaluronic acid compositions.

### SUMMARY

The present disclosure broadly includes crosslinked hyaluronic acid compositions. In one aspect, the present disclosure includes hyaluronic acid crosslinked with at least one biologically active compound and compositions comprising same. In an embodiment of the present disclosure, the at least one biologically active compound is selected from amino acids, amino esters, hydroxy acids, hydroxy esters, vitamins, stabilizers and mixtures thereof. In a further embodiment of the present disclosure, the amino acid is selected from lysine, glycine, valine, proline and cysteine. In a further embodiment of the present disclosure, the vitamin is vitamin C.

In an embodiment, the present disclosure includes a crosslinked hyaluronic acid composition comprising:

from 0.5 to 2.0% w/w of hyaluronic acid; and

from 0.1 to 5.0% w/w of at least one biologically active compound; wherein at least a portion of the biologically active compounds are crosslinked with the hyaluronic acid.

In an embodiment, the present disclosure includes injectable hyaluronic acid compositions wherein the hyaluronic acid is crosslinked using crosslinking agents selected from amino acids, amino esters, hydroxy acids, hydroxy esters, vitamins, stabilizers and mixtures thereof. In a further embodiment of the present disclosure, the amino acid is selected from lysine, glycine, valine and proline. In a further embodiment of the present disclosure, the vitamin is vitamin C.

In an embodiment, the present disclosure includes injectable cosmetic compositions comprising crosslinked hyaluronic acid. In a further embodiment of the present disclosure, the hyaluronic acid is crosslinked using crosslinking agents selected from amino acids, amino esters, hydroxy acids, hydroxy esters, vitamins, stabilizers and mixtures thereof. In a further embodiment of the present disclosure, the amino acid is selected from lysine, glycine, valine and proline. In a further embodiment of the present disclosure, the vitamin is vitamin C. In an aspect, the injectable cosmetic compositions provide for prolonged action following injection.

In an embodiment, the present disclosure includes injectable cosmetic compositions comprising crosslinked hyaluronic acid for use in the targeted delivery of biologically active compounds to specific areas of the skin and joints. In a further embodiment of the present disclosure, the biologically active compounds are selected from amino acids, amino esters, hydroxy acids, hydroxy esters, vitamins, stabilizers and mixtures thereof. In a further embodiment of the present disclosure, the amino acid is selected from lysine, glycine, valine and proline. In a further embodiment of the present disclosure, the vitamin is vitamin C.

In an embodiment, the present disclosure includes injectable cosmetic compositions comprising crosslinked hyaluronic acid for use in increasing the metabolic activity of skin. In an aspect, the cosmetic composition activates the synthesis of some of the chief components of the extracellular matrix (ECM), such as hyaluronic acid, collagen, elastin and laminin.

In an embodiment, the present disclosure includes injectable crosslinked hyaluronic acid compositions for use in treating skin conditions, non-limiting examples of which include fine lines or wrinkles, skin that has reduced elasticity, loose skin, skin that is deficient in hyaluronic acid production and skin that is deficient in matrix protein production (e.g. fibronectin, laminin, collagen I, collagen III, and/or elastin).

In an embodiment, the present disclosure includes injectable crosslinked hyaluronic acid compositions for use as a dermal filler.

In an embodiment, the present disclosure includes injectable crosslinked hyaluronic acid compositions for use in treating joint conditions, non-limiting examples of which include osteoarthritis. Such treatments, called viscosupplementation, typically involve injections into the joint and are believed to supplement the viscosity of the joint fluid, thereby lubricating the joint, cushioning the joint, and producing an analgesic effect.

In an embodiment, the present disclosure includes injectable crosslinked hyaluronic acid compositions that are biocompatible.

In an embodiment, the present disclosure includes injectable crosslinked hyaluronic acid compositions that exhibit improved resistance to enzymatic degradation (i.e., increased in vivo half-life) and hence improved bioavailability. Improved resistance to enzymatic degradation provides for a more efficient delivery of the hyaluronic acid as well as the one or more biologically active compounds that are crosslinked with the hyaluronic acid to the cells and tissues where the compositions are delivered and that get released following enzymatic degradation of the hyaluronic acid.

In an embodiment, the present disclosure includes a method for treating a skin condition, the method comprising injecting a composition comprising a crosslinked hyaluronic acid into an area of the skin in need of treatment. In an embodiment of the present disclosure, the hyaluronic acid is crosslinked with at least one biologically active compound. In a further embodiment of the present disclosure, the at least one biologically active compound is selected from amino acids, amino esters, hydroxy acids, hydroxy esters, vitamins, stabilizers and mixtures thereof. In a further embodiment of the present disclosure, the amino acid is selected from lysine, glycine, valine and proline. In yet a further embodiment of the present disclosure, the vitamin is vitamin C. Non-limiting examples of skin conditions include fine lines or wrinkles, skin that has reduced elasticity, loose skin, skin that is deficient in hyaluronic acid production and skin that is deficient in matrix protein production (e.g. fibronectin, laminin, collagen I, collagen III, and/or elastin).

In an embodiment, the present disclosure includes a method for treating a joint condition, the method comprising injecting a composition comprising a crosslinked hyaluronic acid into a joint in need of treatment. In an embodiment of the present disclosure, the hyaluronic acid is crosslinked with at least one biologically active compound. In a further embodiment of the present disclosure, the at least one biologically active compound is selected from amino acids, amino esters, hydroxy acids, hydroxy esters, vitamins, stabilizers and mixtures thereof. In a further embodiment of the present disclosure, the amino acid is selected from lysine, glycine, valine and proline. In yet a further embodiment of the present disclosure, the vitamin is vitamin C. A non-limiting example of a joint condition includes osteoarthritis.

In an embodiment, the present disclosure includes a process for producing a crosslinked hyaluronic acid, the process comprising:
mixing hyaluronic acid with at least one biologically active compound to produce a mixture; and
feeding the mixture into an extruder; and
extruding the mixture under conditions to produce the crosslinked hyaluronic acid.

In an embodiment, the present disclosure includes a process for producing a crosslinked hyaluronic acid, the process comprising:
adding hyaluronic acid and at least one biologically active compound into an extruder; and
co-extruding the hyaluronic acid and the at least one biologically active compound under conditions to produce the crosslinked hyaluronic acid.

In an embodiment of the present disclosure, the at least one biologically active compound is selected from amino acids, amino esters, hydroxy acids, hydroxy esters, vitamins, stabilizers and mixtures thereof. In a further embodiment of the present disclosure, the amino acid is selected from lysine, glycine, valine and proline. In a further embodiment of the present disclosure, the vitamin is vitamin C.

In an embodiment of the present disclosure, the extrusion process is performed using a twin screw extruder (TSE).

The foregoing and other advantages and features of the present disclosure will become more apparent upon reading of the following non-restrictive description of illustrative embodiments thereof, given by way of example only with reference to the accompanying drawings/figures.

### BRIEF DESCRIPTION OF THE DRAWINGS/FIGURES

In the appended drawings/figures:

FIG. 1 is an IR spectrum of hyaluronic acid; Vitamin C (Na ascorbyl phosphate); a mixture of hyaluronic acid and vitamin C (“mechanical mixture”); and an extruded mixture composed of hyaluronic acid and vitamin C (“mixture after extruder”) in accordance with an embodiment of the present disclosure.

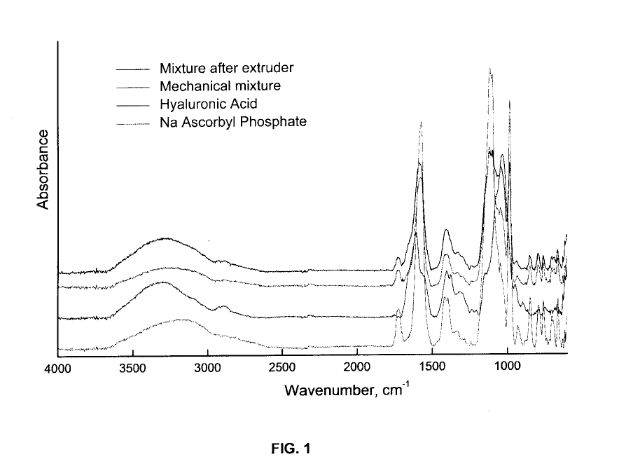

FIG. 2 illustrates an expanded portion of a region (1800 cm⁻¹ to 1200 cm⁻¹) of the IR spectrum of FIG. 1 of hyaluronic acid; Vitamin C (Na ascorbyl phosphate); a mixture of hyaluronic acid and vitamin C (“mechanical mixture”); and an extruded mixture composed of hyaluronic acid and vitamin C (“mixture after extruder”) in accordance with an embodiment of the present disclosure. The spectra illustrate that the interaction between the hyaluronic acid and vitamin C is much stronger for an extruded mixture than for a mechanical mixture as a result of stronger H-bonding. The IR spectrum obtained for the extruded mixture typically shows a λₘₐₓ shift to lower field as compared to a mechanical mixture confirming that the H-bonds are significantly stronger in the extruded mixture.

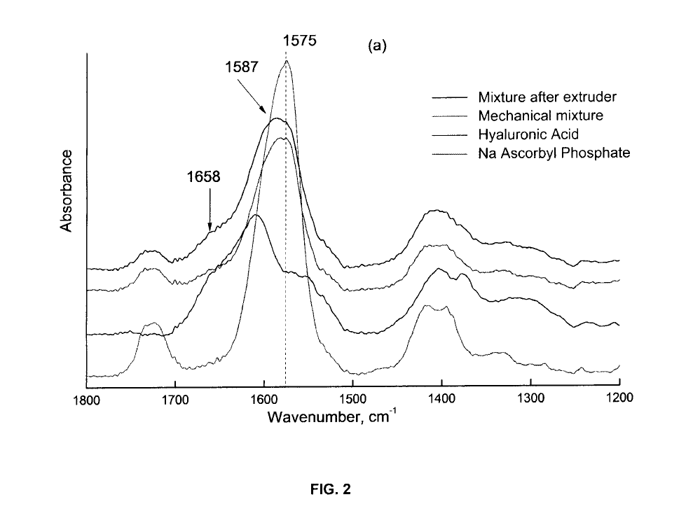

FIG. 3 illustrates an expanded portion of a region (1200 cm⁻¹ to 800 cm⁻¹) of an IR spectrum of FIG. 1 of hyaluronic acid; Vitamin C (Na ascorbyl phosphate); a mixture of hyaluronic acid and vitamin C (“mechanical mixture”); and an extruded mixture composed of hyaluronic acid and vitamin C (“mixture after extruder”) in accordance with an embodiment of the present disclosure. The spectra illustrate that the interaction between the hyaluronic acid and vitamin C is much stronger for an extruded mixture than for a mechanical mixture as a result of stronger H-bonding. The IR spectrum obtained for the extruded mixture typically shows a λₘₐₓ shift to lower field as compared to a mechanical mixture confirming that the H-bonds are significantly stronger in the extruded mixture.

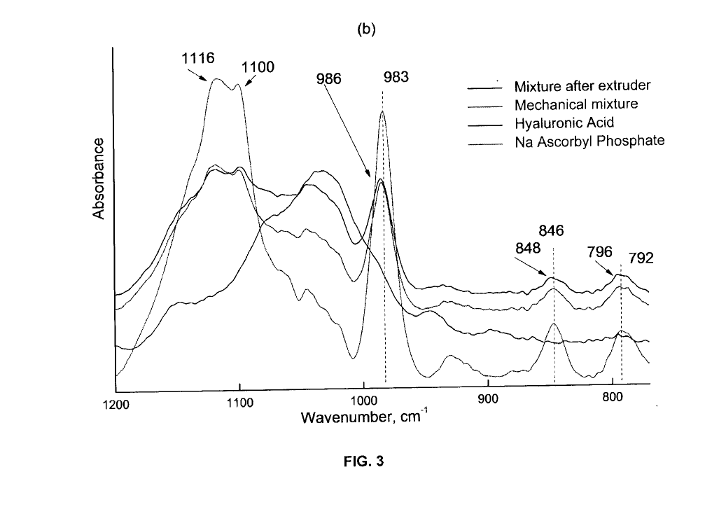

FIG. 4A illustrates a twin screw extruder (TSE) for use in a process in accordance with an embodiment of the present disclosure. FIG. 4B illustrates a section of the screw elements of the twin screw extruder illustrated in FIG. 4A.

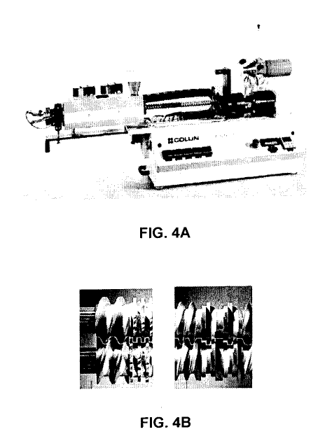

### DETAILED DESCRIPTION

### I. Glossary

In order to provide a clear and consistent understanding of the terms used in the present disclosure, a number of definitions are provided below. Moreover, unless defined otherwise, all technical and scientific terms as used herein have the same meaning as commonly understood by one of ordinary skill in the art to which this specification pertains.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±1% of the modified term if this deviation would not negate the meaning of the word it modifies.

The term “extruder”, as used herein, is intended to refer to any conventional single or double screw extrusion device.

The term “residence time” in an extruder refers to the time taken by a material to get through the extruder, from the feed port to the die. The residence time is measured by adding a small quantity of material containing a coloring agent into the feed port. The chronometer is started when the colorant enters the barrel and is stopped when coloration is observed at the die exit.

The term “extrudate temperature” refers to the temperature of the material at the die exit of an extruder as measured by a portable thermocouple plunged into one of the die openings.

The term “amino acid” as used herein refers to any one of the following L- or D-amino acids: isoleucine, alanine, leucine, asparagine, lysine, aspartic acid, methionine, cysteine, phenylalanine, glutamic acid, threonine, glutamine, tryptophan, glycine, valine, proline, arginine, serine, histidine, and tyrosine.

The term “amino ester” as used herein refers to an alkyl, aryl, or arylalkyl ester of an amino acid.

The term “hydroxy acid” as used herein refers to an organic compound that is functionalized, at least, with both a hydroxy group and a carboxylic acid group. In an embodiment, the hydroxyl acid is an alpha hydroxyl acid, where the hydroxyl group is bonded to the carbon adjacent to the carboxylic acid group.

The term “hydroxy ester” as used herein refers to an alkyl, aryl, or arylalkyl ester of a hydroxyl acid.

The term “vitamin” as used herein refers to any of the common nutrients required by an organism that are generally provided in an organism's diet and includes, for example, vitamins A, B₁, B₂, B₃, B₅, B₆, B₇, B₉, B₁₂, D, E, H and K.

The term “crosslinking” as used herein refers to a process, in which at least two molecules, or two portions of a long molecule, are joined together by a chemical interaction. Such interactions occur in many different ways including, for example, formation of a covalent bond, formation of hydrogen bonds, and/or hydrophobic, hydrophilic, ionic and/or electrostatic interaction. In further examples, molecular interaction is also characterized by an at least temporary physical connection between at least one molecule with itself or between two or more molecules. Therefore, it is contemplated that, in an embodiment, a hyaluronic acid according to the present disclosure crosslinks with itself.

The term “at least a portion” as used herein means that the entire amount of biologically active (i.e. amino acids, amino esters, hydroxy acids, hydroxy esters, vitamins, stabilizers and mixtures thereof) need not be crosslinked with the hyaluronic acid, so long as a portion of the biologically active is crosslinked. For example, a portion may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% or any range derivable therein.

The term “reactive group” as used herein refers to any elements or combinations of elements having sufficient reactivity to be used in crosslinking or coupling with hyaluronic acid. Non-limiting examples of “reactive groups” in include hydroxy, carboxy, amino and thio.

As used herein, the term “stabilizing” includes maintaining a compound in a specific state and preventing or slowing fluctuations from that particular state into another.

As used herein, the terms “stabilizer,” or “preservative” include an agent that prevents the oxidation of other compounds. Examples of preservatives useful in the compositions of the present disclosure include, but are not limited to, an antioxidant, alpha-lipoic acid, 1-carnitine, phenoxyethanol, butylated hydroxytoluene and sodium benzoate. In an embodiment of the present disclosure, the antioxidant includes glutathione. One of ordinary skill in the art will appreciate that other preservatives are useful in the injectable crosslinked hyaluronic acid compositions of the present disclosure. When a preservative is present, it is typically present in an amount of from about 0.1% to about 1.5% by weight.

As used herein, “cosmetic” is an adjective referring to improving the health and/or appearance of a surface or covering defects. Typically, cosmetic compositions are used to improve aesthetic rather than functional aspects of a surface. Most commonly, cosmetic compositions are formulated for application as a health and beauty treatment or for affecting personal appearance of the body, for example, keratinous surfaces such as skin, hair, nails, and the like.

As used herein, the term “derivative” refers to a structural analog and designates a compound having a structure similar to that of another one, but differing from it in respect of a certain component. It can differ in one or more atoms, functional groups, or substructures, which are replaced with other atoms, groups, or substructures. A structural analog can be imagined to be formed, at least theoretically, from the other compound.

As used herein, the term “biologically active” refers to the ability to mediate a biological function. As used herein, the term “biologically active compound” refers to compounds that mediate a biological function and that comprise at least reactive group that participates in crosslinking with the hyaluronic acid.

As used herein, the term “prolonged action” refers to long acting formulations, that is, formulations that have pharmacokinetic characteristics such that the formulation provides for an extended length of release time than is normally found for the released drug itself.

### II Compositions of the Disclosure

The hyaluronic acid of the present disclosure is crosslinked with at least one biologically active compound to form a less water soluble polymer hydrogel network that is more resistant to enzymatic degradation (stabilization effect), and thus requires less frequent reinjection, than the non-crosslinked hyaluronic acid compositions. Suitable biologically active compounds include actives such as amino acids, amino esters, hydroxy acids, hydroxy esters, vitamins and stabilizers. In an embodiment of the present disclosure, the crosslinking is performed using at least one of a vitamin, an amino acid, a hydroxy acid and a stabilizer. In yet a further embodiment of the present disclosure, the crosslinking is performed in the presence of additional actives such that they become trapped and/or crosslinked within the crosslinked hyaluronic acid network.

In an embodiment of the present disclosure, the crosslinked hyaluronic acid performs as a vehicle providing for prolonged bioavailability of the active(s). In an embodiment of the present disclosure, the crosslinking of the hyaluronic acid with a biologically active compound is achieved by means of an extrusion process. The use of extruders as continuous reactors for processes such as polymerization, polymer modification or compatibilization of polymer blends, involves technologies are well documented in the literature. In the case of reactive extrusion, several organic reactions can be conducted in extruders, including polymerization, grafting, copolymer formation, molecular network formation, crosslinking, functionalization and controlled degradation. In an embodiment of the present disclosure, a corotating intermeshing twin screw extruder (TSE) is used. One of the advantages of using an extruder as a continuous reactor resides in the extrusion process being substantially a solid state chemical process. Accordingly, there is no real need for solvents in an extrusion process. In an embodiment of the present disclosure, water is added to the extrusion process in order to modulate the viscosity of the mixture being extruded. A further advantage of using an extrusion process for producing the crosslinked hyaluronic acid compositions of the present disclosure resides in the observation that extrusion processes typically produce less reaction side products. Furthermore, the extrusion process typically provides good yields of desired crosslinked product and provides for the production of solid products from insoluble and thermo labile starting materials.

In an embodiment, the present disclosure includes a crosslinked hyaluronic acid composition comprising:

from 0.5 to 2.0% w/w of hyaluronic acid; and

from 0.1 to 5.0% w/w of at least one biologically active compound; wherein at least a portion of the biologically active compounds are crosslinked with the hyaluronic acid.

In an embodiment, the crosslinked hyaluronic acid composition comprises only a single type of biologically active compound.

In an embodiment, the crosslinked hyaluronic acid composition comprises a mixture of different types of biologically active compound.

In an embodiment of the present disclosure, the compositions comprise from about 0.5% to about 2.0% w/w of hyaluronic acid. In an embodiment, the compositions comprise from about 1.0% to about 2.0% w/w of hyaluronic acid. In a further embodiment, the compositions comprise from about 1.0% to about 1.5% w/w of hyaluronic acid.

In an embodiment, the compositions of the present disclosure comprise one or more vitamins. Illustrative embodiments of vitamins include Vitamin A (retinol), Vitamin B2, Vitamin B3 (niacinamide), Vitamin B6, Vitamin B9, Vitamin C, Vitamin E and Vitamin H (biotin). In an embodiment, derivatives of the vitamins are used. Non-limiting examples of such derivatives include ascorbyl tetraisopalmitate, magnesium ascorbyl phosphate and ascorbyl glucoside, tocopheryl acetate, tocopheryl palmitate and tocopheryl linoleate. In an embodiment, at least a portion of the one or more vitamins is crosslinked to the hyaluronic acid.

In an embodiment of the present disclosure, the compositions comprise from about 0.1% to about 2.0% w/w of one or more vitamins. In an embodiment, the compositions comprise from about 0.1% to about 1.5% w/w of one or more vitamins. In an embodiment, the compositions comprise from about 0.1% to about 1.2% w/w of one or more vitamins. In an embodiment, the compositions comprise from about 0.1% to about 0.5% w/w of one or more vitamins. In a further embodiment, the compositions comprise from about 0.1% to about 0.4% w/w of one or more vitamins.

In an embodiment, the compositions of the present disclosure comprise an amino acid. In an embodiment, the compositions of the present disclosure comprise one or more L- and/or D amino acids. Illustrative examples of amino acids include isoleucine, alanine, leucine, asparagine, lysine, aspartate, methionine, cysteine, phenylalanine, glutamate, threonine, glutamine, tryptophan, glycine, valine, proline, arginine, serine, histidine, and tyrosine. In a further embodiment, the amino acids are selected from lysine, glycine, valine, proline and cysteine. In an embodiment, at least a portion of the one or more amino acids is crosslinked to the hyaluronic acid.

In an embodiment of the present disclosure, the compositions comprise from about 0.1% to about 2.0% w/w of one or more amino acids. In an embodiment, the compositions comprise from about 0.1% to about 1.5% w/w of one or more amino acids. In an embodiment, the compositions comprise from about 0.1% to about 1.2% w/w of one or more amino acids. In an embodiment, the compositions comprise from about 0.1% to about 0.5% w/w of one or more amino acids. In a further embodiment, the compositions comprise from about 0.1% to about 0.4% w/w of one or more amino acids.

In an embodiment, the compositions of the present disclosure comprise a stabilizer. Illustrative examples of stabilizers include L- or D carnitine and glutathione. In an embodiment, at least a portion of the one or more stabilizers is crosslinked to the hyaluronic acid. In an embodiment of the present disclosure, the compositions comprise from about 0.1% to about 2.0% w/w of stabilizer. In an embodiment, the compositions comprise from about 0.1% to about 1.5% w/w of stabilizer. In a further embodiment, the compositions comprise from about 0.1% to about 1.0% w/w of stabilizer.

In an embodiment, the compositions of the present disclosure comprise a hydroxy acid. Non-limiting examples of hydroxy acids include glycolic acid, malic acid, lactic acid, mandelic acid, phytic acid, salicylic acid, aleuritic acid, tartaric acid, citric acid, hydroxytetronic acid, glucuronic acid, mucic acid, galacturonic acid, gluconic acid, saccharic acid, glucoheptonic acid, alpha-hydroxybutyric acid, tartronic acid, alpha-hydroxyisobutyric acid, isocitric acid, alpha-hydroxyisocaproic acid, dihydroxymaleic acid, alpha-hydroxyisovaleric acid, dihydroxytartaric acid, beta-hydroxybutyric acid, dihydroxyfumaric acid, beta-phenyllactic acid, atrolactic acid, galactonic acid, pantoic acid and glyceric acid. In an embodiment, a combination of hydroxy acids is used. In a further embodiment, derivatives of the hydroxy acids are used. In a further embodiment of the present disclosure, the hydroxy acid is tartronic acid. In an embodiment, at least a portion of the one or more hydroxyl acids is crosslinked to the hyaluronic acid.

In an embodiment of the present disclosure, the compositions comprise from about 0.1% to about 1.0% w/w of hydroxy acid. In an embodiment, the compositions comprise from about 0.1% to about 0.8% w/w of hydroxy acid. In an embodiment, the compositions comprise from about 0.1% to about 0.5% w/w of hydroxy acid.

One drawback of administering exogenous hyaluronic acid for therapeutic or other biomedical purposes is that hyaluronic acid degrades very rapidly and consequently loses its viscosity and its lubricity. Crosslinking the hyaluronic acid with at least one biologically active compound generates a less water soluble polymer hydrogel network that is more resistant to enzymatic degradation. The presence of one or more hydrophilic polymers further improves the stability of the hyaluronic acid. Illustrative examples of hydrophilic polymers include hydroxy polyanionic polysaccharides such as sodium alginate, alginic acid, propylene glycol alginate, carboxymethyl cellulose and carboxyethyl cellulose. In an embodiment, at least a portion of the one or more hydrophilic polymers is crosslinked to the hyaluronic acid.

In an embodiment, the compositions of the present disclosure comprise a hydrophilic polymer. In an embodiment of the present disclosure, the compositions comprise from about 0.5% to about 2.0% w/w of hydrophilic polymer. In an embodiment, the compositions comprise from about 1.0% to about 2.0% w/w of hydrophilic polymer. In a further embodiment, the compositions comprise from about 1.2% to about 1.7% w/w of hydrophilic polymer. In yet a further embodiment, the hydrophilic polymer is carboxymethylcellulose.

In an embodiment, the compositions of the present disclosure optionally include, as the remainder of the composition, a pharmaceutically acceptable carrier. In an embodiment, the pharmaceutically acceptable carrier facilitates processing the crosslinked hyaluronic acid into pharmaceutically acceptable compositions. As used herein, the term “pharmacologically acceptable carrier” is synonymous with “pharmacological carrier” and refers to any carrier that has substantially no long term or permanent detrimental effect when administered to subjects including humans and encompasses terms such as “pharmacologically acceptable vehicle, stabilizer, diluent, additive, auxiliary, or excipient.” A carrier generally is mixed with an active compound or permitted to dilute or enclose the active compound and is for example a solid, semi-solid, or liquid agent. In the context of the present disclosure, the active compound in the crosslinked hyaluronic acid. It is understood that the active compound is soluble or is delivered as a suspension in the desired carrier or diluent. In an embodiment of the present disclosure, the carrier or diluent includes water, saline and the like.

### III. Uses and Methods of the Disclosure

The hyaluronic acid of the present disclosure is crosslinked with at least one biologically active compound to form a less water soluble polymer hydrogel network that is more resistant to enzymatic degradation (stabilization effect). In an aspect, the present disclosure includes administering a hydrogel composition to an individual having a skin condition. As used herein, the term “administering” refers to delivering a composition comprising a crosslinked hyaluronic acid to an individual and which administration potentially results in a clinically, therapeutically, or experimentally beneficial result.

The actual delivery mechanism and dosage regimen is readily determined by a person of ordinary skill in the art by taking into account factors, including, without limitation, the type of skin condition, the location of the skin condition, the cause of the skin condition, the severity of the skin condition, the degree of relief desired, the duration of relief desired, the particular composition used, the pharmacodynamics of the particular composition used, the nature of the other compounds included in the particular composition used, the particular route of administration, the particular characteristics, history and risk factors of the individual, such as, e.g., age, weight, general health and the like, or any combination thereof. In an embodiment, the compositions of the present disclosure are administered to a skin region of an individual by injection.

In an embodiment of the present disclosure, the crosslinked hyaluronic acid is a delivery vehicle for a biologically active compound crosslinked with the hyaluronic acid. A delivery vehicle includes a sustained release delivery vehicle. In an embodiment, the term “sustained release” refers to the release of a biologically active compound over a period of at least one day. In an embodiment, such a delivery vehicle achieves a controlled active release profile of the biologically active compound over time. In a further embodiment, the release profile of the biologically active compound is dependent on the enzymatic degradation of the hyaluronic acid. In a further embodiment, at least a portion of the total amount of the biologically active compound is crosslinked with the hyaluronic acid. In an embodiment, crosslinking the biologically active compound with the hyaluronic acid provides for prolonged bioavailability of the biologically active compound. In an embodiment of the present disclosure, the biologically active compound is selected from amino acids, amino esters, hydroxy acids, hydroxy esters, vitamins, stabilizers and mixtures thereof.

The crosslinked hyaluronic acid compositions of the present disclosure are injectable using minimally invasive procedures. Such local administration at the target site provides a high level of concentration of the hyaluronic acid and the biologically active crosslinked therewith. Furthermore, local administration minimizes potential adverse systemic effects of the compositions.

In an embodiment, the present disclosure includes a process for producing a crosslinked hyaluronic acid, the process comprising:
mixing hyaluronic acid with at least one biologically active compound to produce a mixture; and
feeding the mixture into an extruder; and
extruding the mixture under conditions to produce the crosslinked hyaluronic acid.

In an embodiment, the present disclosure also includes a process for producing a crosslinked hyaluronic acid, the process comprising:
adding hyaluronic acid and at least one biologically active compound into an extruder; and
co-extruding the hyaluronic acid and the at least one biologically active compound under conditions to produce the crosslinked hyaluronic acid.

In an embodiment of the present disclosure, the mixture is prepared by thoroughly mixing hyaluronic acid with at least one biologically active compound. Biologically active compounds include amino acids, amino esters, hydroxy acids, hydroxy esters, vitamins, stabilizers and mixtures thereof. When a substantially homogeneous mixture is obtained, the mixture is fed into an extruder resulting in a hyaluronic acid that is crosslinked with the at least one biologically active compound. In an embodiment of the present disclosure, the extruder is a twin screw extruder. In an embodiment, the substantially homogeneous mixture is extruded at a temperature of about 20° C. to about 40° C., or at about 25° C. to about 30° C., at a pressure of about 1 MPa to about 10 MPa, or about 5 MPa, with a residence time of about 5 minutes to about 30 minutes, or about 8 minutes to about 15 minutes.

### EXPERIMENTAL

A number of examples are provided herein below illustrating the preparation of various crosslinked hyaluronic acid compositions in accordance with the present disclosure. The following non-limiting examples are illustrative of the present disclosure.

All compositions are presented as sterile aqueous solutions in the presence of sodium chloride (1-2%/wt.) and phosphate buffer pH 7.4 (1%/wt.). The solutions are typically preserved as ampules, cartridges, vials or prefillable syringes.

Non-limiting examples of various skin treatment formulations.

### Example 1

### Composition 1

A mixture of hyaluronic acid Na salt (100 g), carboxymethylcellulose sodium salt (150 g) and magnesium ascorbylphosphate (50 g) were thoroughly mixed in a suitable vessel. The resulting mixture was subsequently fed into a twin screw extruder (Bench Type Compounder ZK 25T) subjecting the mixture to both pressure and shearing forces. The extruder operating temperature was set at 25-30° C. whereas the pressure exerted on the mixture by the co-rotating screws was 5 MPa. The residence time of the mixture was about 10 minutes. The amount of crosslinked hyaluronate product recovered from the extruder was measured to be 294 g (98%).

The resulting viscous product was subsequently mixed with sodium chloride (between 100 to 200 g) and phosphate buffer (pH 7.4; 100 g) and then reconstituted in distilled water up to a total volume of 10 L. Following degassing, the homogeneous solution was distributed in glass syringes (sizes ranging from 0.5 mL to 3.0 mL) or glass vials (sizes ranging from 0.5 mL to 20.0 mL). Finally, the syringes and/or vials were sterilized under reduced pressure at a temperature of 120° C. over a period of about 45 minutes. The characteristics of the sterilized product are reported in Table 1.

### Example 2

### Compositions 2-8

A mixture of hyaluronic acid Na salt (140 g), magnesium ascorbylphosphate (40 g), glycine (30 g), proline (30 g) and lysine HCl (30 g) were thoroughly mixed in a suitable vessel. The resulting mixture was subsequently fed into a twin screw extruder (Bench Type Compounder ZK 25T) subjecting the mixture to both pressure and shearing forces. The extruder operating temperature was set at 25-30° C. whereas the pressure exerted on the mixture by the co-rotating screws was 5 MPa. The residence time of the mixture was about 10 minutes. The amount of crosslinked hyaluronate product recovered from the extruder was measured to be 262 g (97%).

The resulting viscous product was subsequently mixed with sodium chloride (between 100 to 200 g) and phosphate buffer (pH 7.4; 100 g) and then reconstituted in distilled water up to a total volume of 10 L. Following degassing, the homogeneous solution was distributed in glass syringes (sizes ranging from 0.5 mL to 3.0 mL) or glass vials (sizes ranging from 0.5 mL to 20.0 mL). Finally, the syringes and/or vials were sterilized under reduced pressure at a temperature of 120° C. over a period of about 45 minutes. The characteristics of the sterilized product are reported in Table 2.

Non-limiting examples of various compositions (1-8) in accordance with embodiments of the present disclosure are reported in Tables 3-10.

### Example 3

### Efficacy of Compositions on Skin Moisture and Skin Elasticity

Studies were performed on female panelists. These studies included all of the compositions described in Tables 3-10. The study parameters included injecting the compositions into the panelist's skin in a regimen-like format. The effectiveness of this regimen was tested on 27 female volunteers exhibiting various degrees of dry facial skin and levels of wrinkling. The compositions were injected into various areas including the face, neck and lower neck according to the following regimen: t=0 (1^{st} injection); t=14 days (2^{nd} injection); t=21 days (3^{rd} injection); and t=28 days (4^{th} injection). Typical injection volumes ranged from 1 to 3 mL depending on the severity of the condition. For all panelists, the skin moisture showed an increase of 12-15% following the 4^{th} injection. An increase in the skin's elasticity of about 8% was observed in 24 panelists following the 4^{th} injection; and 2 panelists showed an increase of about 4% following the 4^{th} injection. The skin pH remained constant for all panelists during the course of the study.

Non-limiting examples of various joint treatment compositions (9-11) in accordance with embodiments of the present disclosure are reported in Tables 11-13.

### Example 4

### Efficacy of Compositions on Joint Treatment

Studies were performed on 12 panelists (8 males and 4 females). Seven (7) panelists were suffering from gonarthrosis whereas four (4) panelists were suffering from coxarthrosis. These studies included all of the compositions described in Tables 11-13. The study parameters included injecting the compositions into the panelist's knee and ankle in a regimen-like format. The compositions were injected according to the following regimen: t=0 (1^{st} injection); t=14 days (2^{nd} injection); t=21-28 days (3^{rd} injection); and t=42-56 days (4^{th} injection). Typical injection volumes ranged from 0.5 to 20 mL depending on the severity of the condition. For all panelists, pain was reduced and range of motion increased shortly after the injection. Three (3) to five (5) days following the injection, no significant reoccurrence of pain and inflammation could be observed.

### Example 5

### Efficacy of Compositions on Joint Treatment and Inflammation

Studies were performed on 9 panelists. Four (4) panelists were post-surgery panelists; two (2) panelists were suffering from synovitis; whereas three (3) panelists were suffering from bursitis. These studies included all of the compositions described in Tables 11-13. The study parameters included injecting the compositions into the panelist's area of inflammation in a regimen-like format. The compositions were injected according to the following regimen: t=0 (1^{st} injection); t=14 days (2^{nd} injection); t=21-28 days (3^{rd} injection); and t=42-56 days (4^{th} injection). Typical injection volumes ranged from 0.5 to 20 mL depending on the severity of the condition. For all panelists, pain was reduced and range of motion increased shortly after the injection.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

TABLE 1
Appearance and Physical Characteristics

| Appearance | Transparent Gel-Like Substance |
|---|---|
| Dynamic Viscosity (mPa s) | 15000-25000 |
| pH | 6.0-8.0 |
| Total Volume | 0.5-20 mL |

TABLE 2
Appearance and Physical Characteristics

| Appearance | Transparent Gel-Like Substance |
|---|---|
| Dynamic Viscosity (mPa s) | 3000-7000 |
| pH | 6.0-8.0 |
| Total Volume | 0.5-20 mL |

TABLE 3
Product Composition
Product Composition 1- Example 1

| Component | % Wt | Weight (g) |
|---|---|---|
| Hyaluronate Sodium Salt | 1.0 | 100 |
| Carboxymethylcellulose Sodium | 1.5 | 150 |
| Salt | | |
| Magnesium Ascorbylphosphate | 0.5 | 50 |
| NaCl | 1.0-2.0 | 100-200 |
| Phosphate Buffer (pH 7.4) | 1.0 | 100 |
| H₂O | Residual | Up to 10 L |

TABLE 4
Product Composition
Product Composition 2 - Example 2

| Component | % Wt | Weight (g) |
|---|---|---|
| Hyaluronate Sodium Salt | 1.4 | 140 |
| Magnesium Ascorbylphosphate | 0.4 | 40 |
| Glycine | 0.3 | 30 |
| L-Proline | 0.3 | 30 |
| L-Lysine monochloride | 0.3 | 30 |
| NaCl | 1.0-2.0 | 100-200 |
| Phosphate Buffer (pH 7.4) | 1.0 | 100 |
| H₂O | Residual | Up to 10 L |

TABLE 5
Product Composition
Product Composition 3 - Example 2

| Component | % Wt | Weight (g) |
|---|---|---|
| Hyaluronate Sodium Salt | 1.4 | 140 |
| Magnesium Ascorbylphosphate | 0.4 | 40 |
| L-Carnitine | 1.0 | 100 |
| NaCl | 1.0-2.0 | 100-200 |
| Phosphate Buffer (pH 7.4) | 1.0 | 100 |
| H₂O | Residual | Up to 10 L |

TABLE 6
Product Composition
Product Composition 4 - Example 2

| Component | % Wt | Weight (g) |
|---|---|---|
| Hyaluronate Sodium Salt | 1.4 | 140 |
| Magnesium Ascorbylphosphate | 0.4 | 40 |
| Glycine | 0.3 | 30 |
| L-Valine | 0.3 | 30 |
| L-Cysteine | 0.3 | 30 |
| NaCl | 1.0-2.0 | 100-200 |
| Phosphate Buffer (pH 7.4) | 1.0 | 100 |
| H₂O | Residual | Up to 10 L |

TABLE 7
Product Composition
Product Composition 5 - Example 2

| Component | % Wt | Weight (g) |
|---|---|---|
| Hyaluronate Sodium Salt | 1.4 | 140 |
| Magnesium Ascorbylphosphate | 0.4 | 40 |
| Folic Acid (Vitamin B9) | 0.1 | 10 |
| NaCl | 1.0-2.0 | 100-200 |
| Phosphate Buffer (pH 7.4) | 1.0 | 100 |
| H₂O | Residual | Up to 10 L |

TABLE 8
Product Composition
Product Composition 6 - Example 2

| Component | % Wt | Weight (g) |
|---|---|---|
| Hyaluronate Sodium Salt | 1.4 | 140 |
| Magnesium Ascorbylphosphate | 0.4 | 40 |
| Glutathione | 0.5 | 50 |
| NaCl | 1.0-2.0 | 100-200 |
| Phosphate Buffer (pH 7.4) | 1.0 | 100 |
| H₂O | Residual | Up to 10 L |

TABLE 9
Product Composition
Product Composition 7 - Example 2

| Component | % Wt | Weight (g) |
|---|---|---|
| Hyaluronate Sodium Salt | 1.4 | 140 |
| Magnesium Ascorbylphosphate | 0.4 | 40 |
| Tartronic Acid | 0.5 | 50 |
| NaCl | 1.0-2.0 | 100-200 |
| Phosphate Buffer (pH 7.4) | 1.0 | 100 |
| H₂O | Residual | Up to 10 L |

TABLE 10
Product Composition
Product Composition 8 - Example 2

| Component | % Wt | Weight (g) |
|---|---|---|
| Hyaluronate Sodium Salt | 1.2 | 120 |
| Carboxymethylcellulose Sodium | 0.8 | 80 |
| Salt | | |
| Magnesium Ascorbylphosphate | 0.3 | 30 |
| Carnitine | 0.3 | 30 |
| NaCl | 1.0-2.0 | 100-200 |
| Phosphate Buffer (pH 7.4) | 1.0 | 100 |
| H₂O | Residual | Up to 10 L |

TABLE 11
Product Composition
Product Composition 9 - Examples 4-5

| Component | % Wt | Weight (g) |
|---|---|---|
| Hyaluronate Sodium Salt | 1.4 | 140 |
| Magnesium Ascorbylphosphate | 0.4 | 40 |
| Glycine | 0.3 | 30 |
| L-Proline | 0.3 | 30 |
| L-Lysine monochloride | 0.3 | 30 |
| NaCl | 1.0-2.0 | 100-200 |
| Phosphate Buffer (pH 7.4) | 1.0 | 100 |
| H₂O | Residual | Up to 10 L |

TABLE 12
Product Composition
Product Composition 10 - Examples 4-5

| Component | % Wt | Weight (g) |
|---|---|---|
| Hyaluronate Sodium Salt | 1.0 | 100 |
| Magnesium Ascorbylphosphate | 0.4 | 40 |
| Glycine | 0.3 | 30 |
| L-Proline | 0.3 | 30 |
| L-Lysine monochloride | 0.3 | 30 |
| L-Valine | 0.3 | 30 |
| NaCl | 1.0-2.0 | 100-200 |
| Phosphate Buffer (pH 7.4) | 1.0 | 100 |
| H₂O | Residual | Up to 10 L |

TABLE 13
Product Composition
Product Composition 11 - Examples 4-5

| Component | % Wt | Weight (g) |
|---|---|---|
| Hyaluronate Sodium Salt | 1.4 | 140 |
| Magnesium Ascorbylphosphate | 0.4 | 40 |
| Glutathione | 0.5 | 50 |
| L-Cysteine | 0.3 | 30 |
| NaCl | 1.0-2.0 | 100-200 |
| Phosphate Buffer (pH 7.4) | 1.0 | 100 |
| H₂O | Residual | Up to 10 L |

1. A crosslinked hyaluronic acid composition comprising:
from 0.5 to 2.0% w/w of hyaluronic acid; and
from 0.1 to 5.0% w/w of at least one biologically active compound;
wherein at least a portion of the biologically active compounds are crosslinked with the hyaluronic acid.

2. The composition of claim 1, wherein the at least one biologically active compound is selected from amino acids, amino esters, hydroxy acids, hydroxy esters, vitamins and stabilizers.

3. The composition of claim 2, wherein the at least one biologically active compound is a vitamin.

4. The composition of claim 3, wherein the vitamin is selected from at least one of Vitamin A (retinol), Vitamin B2, Vitamin B3 (niacinamide), Vitamin B6, Vitamin B9, Vitamin C, Vitamin E and Vitamin H (biotin).

5. The composition of claim 4, wherein the vitamin comprises from 0.1 to 1.0% w/w of the composition.

6. The composition of claim 3, wherein at least a portion of the vitamin is crosslinked with the hyaluronic acid.

7. The composition of claim 2, wherein the at least one biologically active compound is an amino acid.

8. The composition of claim 7, wherein the amino acid is selected from at least one of isoleucine, alanine, leucine, asparagine, lysine, aspartatic acid, methionine, cysteine, phenylalanine, glutamatic acid, threonine, glutamine, tryptophan, glycine, valine, proline, arginine, serine, histidine, and tyrosine.

9. The composition of claim 8, wherein the amino acid is selected from at least one of lysine, glycine, valine, proline and cysteine.

10. The composition of claim 7, wherein the amino acid comprises from 0.1 to 2.0% w/w of the composition.

11. The composition of claim 7, wherein at least a portion of the amino acid is crosslinked with the hyaluronic acid.

12. The composition of claim 2, wherein the at least one biologically active compound is a stabilizer.

13. The composition of claim 12, wherein the stabilizer is carnitine.

14. The composition of claim 12, wherein the stabilizer is glutathione.

15. The composition of claim 12, wherein the stabilizer comprises from 0.1 to 2.0% w/w of the composition.

16. The composition of claim 12, wherein at least a portion of the stabilizer is crosslinked with the hyaluronic acid.

17. The composition of claim 2, wherein the at least one biologically active compound is a hydroxy acid.

18. The composition of claim 17, wherein the hydroxy acid is selected from at least one of glycolic acid, malic acid, lactic acid, mandelic acid, phytic acid, salicylic acid, aleuritic acid, tartaric acid, citric acid, hydroxytetronic acid, glucuronic acid, mucic acid, galacturonic acid, gluconic acid, saccharic acid, glucoheptonic acid, alpha-hydroxybutyric acid, tartronic acid, alpha-hydroxyisobutyric acid, isocitric acid, alpha-hydroxyisocaproic acid, dihydroxymaleic acid, alpha-hydroxyisovaleric acid, dihydroxytartaric acid, beta-hydroxybutyric acid, dihydroxyfumaric acid, beta-phenyllactic acid, atrolactic acid, galactonic acid, pantoic acid and glyceric acid.

19. The composition of claim 17, wherein the hydroxy acid is tartronic acid.

20. The composition of claim 17, wherein the hydroxy acid comprises from 0.1 to 2.0% w/w of the composition.

21. The composition of claim 17, wherein at least a portion of the hydroxy acid is crosslinked with the hyaluronic acid.

22. The composition of any one of claims 1 to 21, further comprising a hydrophilic polymer.

23. The composition of claim 22, wherein the hydrophilic polymer is carboxymethyl cellulose.

24. The composition of claim 22, wherein the hydrophilic polymer comprises from 0.1 to 2.0% w/w of the composition.

25. The composition of claim 22, wherein at least a portion of the hydrophilic polymer is crosslinked with the hyaluronic acid.

26. The composition of claim 1, comprising:
1.4% w/w of hyaluronic acid;
0.4% w/w vitamin C; and
0.1% w/w vitamin B9.

27. The composition of claim 1, comprising:
1.4% w/w of hyaluronic acid;
0.4% w/w vitamin C;
0.3% w/w glycine;
0.3% w/w proline; and
0.3% w/w lysine.

28. The composition of claim 1, comprising:
1.4% w/w. of hyaluronic acid;
0.4% w/w vitamin C;
0.3% w/w glycine;
0.3%/wt. valine; and
0.3%/wt. cysteine.

29. The composition of claim 1, comprising:
1.4% w/w. of hyaluronic acid;
0.4% w/w vitamin C; and
1.0% w/w carnitine.

30. The composition of claim 1, comprising:
1.4% w/w of hyaluronic acid;
0.4% w/w vitamin C; and
0.5% w/w glutathione.

31. The composition of claim 1, comprising:
1.4% w/w of hyaluronic acid;
0.4% w/w vitamin C; and
0.5% w/w tartronic acid.

32. The composition of claim 1, comprising:
1.0% w/w of hyaluronic acid;
0.5% w/w vitamin C; and
1.5% w/w carboxymethyl cellulose.

33. The composition of claim 1, comprising:
1.2% w/w of hyaluronic acid;
0.3% w/w vitamin C;
0.8% w/w carboxymethyl cellulose; and
0.3% w/w carnitine.

34. The composition of claim 1 comprising:
1.0% w/w of hyaluronic acid;
0.4% w/w vitamin C;
0.3% w/w glycine;
0.3% w/w proline;
0.3% w/w lysine; and
0.3% w/w valine.

35. The composition of claim 1, comprising:
1.4% w/w of crosslinked hyaluronic acid;
0.4% w/w vitamin C;
0.5% w/w glutathione; and
0.3% w/w cysteine.

36. The composition of any one of claims 1 to 35 for use in the treatment of a skin condition.

37. The composition of claim 36, wherein the skin condition is selected from fine lines or wrinkles, skin that has reduced elasticity, loose skin, skin that is deficient in hyaluronic acid production and skin that is deficient in matrix protein production.

38. The composition of any one of claims 1 to 35 for use in the treatment of a joint condition.

39. The composition of claim 38, wherein the joint condition is an anti-inflammatory condition.

40. The composition of claim 39, wherein the joint condition is osteoarthritis.

41. A method for treating a skin condition, the method comprising injecting a composition according to any one of claims 1 to 35 into an area of the skin in need of treatment.

42. The method of claim 41, wherein the skin condition is selected from fine lines or wrinkles, skin that has reduced elasticity, loose skin, skin that is deficient in hyaluronic acid production and skin that is deficient in matrix protein production.

43. A method for treating a joint condition, the method comprising injecting a composition according to any one of claims 1 to 35 into a joint in need of treatment.

44. The method of claim 43, wherein the joint condition is an anti-inflammatory condition.

45. The method of claim 44, wherein the joint condition is osteoarthritis.

46. A process for producing a crosslinked hyaluronic acid, the process comprising:
mixing hyaluronic acid with at least one biologically active compound to produce a mixture; and
feeding the mixture into an extruder; and
extruding the mixture under conditions to produce the crosslinked hyaluronic acid.

47. A process for producing a crosslinked hyaluronic acid, the process comprising:
adding hyaluronic acid and at least one biologically active compound into an extruder; and
co-extruding the hyaluronic acid and the at least one biologically active compound under conditions to produce the crosslinked hyaluronic acid.

48. The process of claim 46 or 47, wherein at least a portion of the biologically active compound is crosslinked with the hyaluronic acid.

49. The process of claim 48, wherein the at least one biologically active compound is selected from amino acids, amino esters, hydroxy acids, hydroxy esters, vitamins and stabilizers.

50. The process of claim 48, wherein the extruder is a twin screw extruder.
